(19) United States
(12) Patent Application Publication
(10) Pub. No.: US 2025/0148390 A1
(43) Pub. Date: May 8, 2025

(54) METHOD FOR PREDICTING RAW MATERIAL FUNCTIONALITY IN THE END PRODUCT

(71) Applicant: DMK Deutsches Milchkontor GmbH, Zeven (DE)

(72) Inventors: Katja BODE, Hollenstedt (DE); Maren Christina SCHULZ, Wilstedt (DE); Ralf ZINK, Bad Zwischenahn (DE)

(21) Appl. No.: 18/908,067

(22) Filed: Oct. 7, 2024

(30) Foreign Application Priority Data
Nov. 8, 2023 (EP) 23 208 547.2

(51) Int. Cl. G06Q 10/0631; G06F 17/18; G06Q 50/02

(52) U.S. Cl. CPC G06Q 10/06315; G06F 17/18; G06Q 50/02

(57) ABSTRACT

A method is proposed for predicting how the incorporation of unknown raw materials will affect the predictable and realizable properties of end products.

### AREA OF THE INVENTION

The present invention is in the field of food technology and relates to a method for predicting the functionality of raw materials in end products, in particular in dairy products, products based primarily and mainly on milk, including cheeses, their plant-based milk alternatives and corresponding hybrid products, by collecting defined analysis and raw material data and evaluating them statistically.

### TECHNOLOGICAL BACKGROUND

The vegan diet trend has arrived in Germany. Around 1.3 million people follow a vegan diet. In comparison, around 8 million eat a vegetarian diet. But the numbers are rising every day. This also increases interest in plant-based raw materials that are suitable substitutes for animal products. In the area of dairy products, the search is on for substitutes for the important milk proteins, among other things. Candidates for this are, for example, vegetable proteins such as those obtained from peas, field beans or oats. However, the following aspect is problematic: milk proteins cannot be exchanged 1:1 for any other type of protein because, on the one hand, the recipes are based on milk as a protein source and, on the other hand, the proteins can have very different structures and functionalities. The same applies to the replacement of animal and/or vegetable macronutrients such as fat and carbohydrates. Another aspect that can affect the raw material functionality in the end product is that the raw material production processes vary depending on the supplier, resulting in different raw material qualities and batches. As a result, when raw materials of the same weight are exchanged, the resulting products may suddenly have a floury, rubbery or dull texture instead of a structured texture, for example.

In order to counteract this, it has so far been necessary to first incorporate potentially suitable candidates into various end products and to check the desired properties that result. In view of the fact that the market now offers hundreds of different raw materials and additives for influencing the end product composition, structure and stability, both on a dairy and plant basis, the necessary pre-selection is extremely time-consuming. It would therefore be desirable to have a procedure that allows rapid prediction of the properties to be expected in the end products.

### STATE OF THE ART

Evaluation methods are known from the prior art, which can be used, for example, to predict the shelf life of food in a package. For example, EP 1626275 B1 (WILD) proposes a method for evaluating product shelf life in a packaging material, which comprises the following steps:
a) introduction of a sealed packaging material (4) filled with filling material into a pressurized chamber (1);
b) storage of the packaging material (4) in the pressurized chamber (1) for a period t1 at excess pressure p1 and a specific temperature T1 in a test gas atmosphere, whereby a defined quantity Q of test gas permeates into the packaging material; and
c) storage of the packaging material (4) for a specific period t2 under the influence of heat and/or light, and d) analytical and/or sensory examination of the filling material.

The disadvantage of the method is the high cost of the apparatus. In addition, the functional principle-simulation of the amount of oxygen introduced into the filling material and acceleration of the reaction by increased pressure-ultimately only allows a rough estimate, which is often unsuitable for a qualitative statement, especially since it is known that for many polymer/gas systems the solubility and diffusion coefficient of the permeating substance in the polymer (e.g. a plastic bottle) depend on the concentration and pressure if the test is carried out below the glass temperature Tg of the polymers and/or the critical temperature of the gas [MÜLLER, K. “Sauerstoff-Durchlässigkeit von Kunststoffflaschen und Verschlüssen” Dissertation TU München (2003)].

A completely different method of visually indicating the shelf life of a packaged foodstuff to the consumer is the subject of property rights EP 2378354 B1, EP 2581785 B1 and WO 2009 056591 A1 (UNI MÜNSTER). These relate to a sensor device, specifically an electrochemical processor, which is short-circuited when the packaging is opened. An aluminum oxide layer is formed, the progression of which is proportional to the shelf life of the product. An increase in temperature, for example, accelerates the formation of oxide and thus naturally also reflects the decreasing degree of freshness of the product. However, this solution makes it necessary to equip each individual package with a separate sensor, which is also technically complex.

EP 3875953 B1 (DMK) describes an accelerated storage test for determining the quality and shelf life of foodstuffs, in which a first sample is stored at a temperature T1 and a similar second sample is stored at a significantly higher temperature T2, whereby the samples are sensory assessed and compared with each other via a calibration curve. Since this sensory evaluation of products always contains a subjective component, a method based exclusively on objective parameters would be more advantageous.

None of these existing test methods deal with the early, targeted prediction of raw material functionality in the end product.

### OBJECT OF THE INVENTION

Therefore, the task of the present invention was to provide a test method which, through the correlated observation and statistical evaluation of different raw material properties, allows the raw material functionality in end products such as milk products, products on a primary and main basis of milk including cheeses, their plant-based milk alternatives and corresponding hybrid products to be reliably predicted at an early stage and in a reproducible manner.

### DESCRIPTION OF THE INVENTION

The invention refers to a method for predicting raw material functionality in end products such as dairy products, primary and main milk-based products including cheese, their plant-based milk alternatives and corresponding hybrid products, comprising or consisting of the following steps
(a) providing a set of raw materials of different qualities;
(b) establishing a set of test parameters;
(c) applying the set of test parameters to each of the individual samples from step (a) to generate a data set;
(d) incorporating the raw materials of different qualities from step (a) into final products, such as dairy products, products based primarily and principally on milk, including cheeses whose plant-based milk alternatives and corresponding hybrid products;
(e) recording and evaluation of product characteristics;
(f) checking the correlation of the data in the data set using Principal Component Analysis (PCA) to obtain a coordinate system (score plot) and determining the largest variance of the data as the first component and the second largest variance of the data as the second component;
(g) assigning the product properties found from step (e) to the data points in the coordinate system from step (f); and
(h) determining clusters in the score plot to identify raw materials which, when used in the end product, lead to the desired similar or identical product properties.

Surprisingly, it was found that with the aid of the method according to the invention, new raw materials can be quickly, reliably and reproducibly assessed in advance with regard to different properties and functionalities that they produce when incorporated into different dairy products, products based primarily and mainly on milk, including cheeses and their plant-based milk alternatives, as well as corresponding hybrid products, on the basis of physico-chemical properties that are predominantly recorded anyway during the initial testing of the raw materials, but are not used for a described prediction.

The method according to the invention is divided into four sections:
selection of suitable analysis parameters
data generation
verification of the correlation of the data by PCA and
determination of clusters in the score plot to identify raw materials that lead to the desired similar or identical product properties when used in the product.
   Dairy Products, Products Based Primarily and Principally on Milk, Including Cheeses or their Plant-Based Milk Alternatives and Corresponding Hybrid Products

The selection of products to which the method according to the invention can be applied is in itself not critical. Typically, the products are selected from the group formed by whole milk, skimmed milk, UHT milk, milk powders, cream, curd, cheese and yoghurt, but also products based primarily and mainly on milk, such as numerous desserts. Also included are their plant-based milk alternatives such as plant-based drinks, spreads, cream alternatives, yogurt alternatives and cheese alternatives. The process can also be applied to hybrid products in which only some of the animal milk components have been replaced with plant-based alternatives or vice versa (replacement of plant-based components with animal-based raw materials or ingredients). This list is not exhaustive.

### Raw Materials

The raw materials whose properties and functionalities are to be predicted in the end products can be selected from the group formed by raw materials such as proteins, fats, carbohydrates, but also semi-finished products such as acid whey, milk permeates and milk retentates, their mixtures and their corresponding plant substitutes or raw materials. They can be in solid, powdered form or also liquid and concentrated.

Usually, a raw material set of 5 to 50, preferably 7 to 15 raw materials is required for the desired prediction of the raw material functionality in the end product, which preferably leads to at least three different end product properties.

### Test Parameters

An essential feature of the method according to the invention is the selection of the test parameters on the basis of which the samples are to be evaluated. The following are preferably considered for this purpose:
pH value;
solubility;
thermal behavior (DSC) of the powder;
particle size distribution (D10, D50, D90, specific surface area);
fat absorption capacity (rapeseed oil);
water absorption capacity;
dissolved oxygen quantity;
instability index (Lumisizer);
lightness color value white/black (L*);
color coordinate red/green (a*);
color coordinate yellow/blue (b*);
free amino nitrogen (PAN);
ammonia content;
urea content;
calcium content;
acidity;
rheological behavior;
firmness; and
degree of syneresis.

The DSC data are the results obtained from differential thermal analysis. This is a thermal analysis method for measuring the amount of heat released or absorbed by a sample during heating, cooling or an isothermal process. An encapsulated container with a sample (5-40 mg) and a second identical container without contents (reference) or a reference sample are exposed together in a temperature chamber to the same temperature change program. As a result of the heat capacity of the sample and any exothermic or endothermic processes or phase changes such as melting or evaporation, there are temperature differences between the sample and the reference, as thermal energy flows into or out of the sample during the process under investigation. The DSC is primarily used to determine the degree of crystallization, the melting or decomposition point and the degree of protein denaturation. Among other things, the temperature at which the thermally induced reaction begins (onset) and ends (final set), the temperature at which the reaction reaches its maximum (peak) and the height of the peak are measured. The reaction enthalpy is also determined.

The specific surface area and the particle size distribution of the powders were determined using static laser light scattering (Horiba LA-960). For the purposes of the method according to the invention, the specific surface area of the powders is a volume-related quantitysᵥ with the unit ^{m2/m3}. The mass-related specific surface area_{SM} is usually determined, as this is easier to carry out experimentally. The two values can be converted using the factor ρ

sv=ρ_{SM}

The particle size distribution comprises the specification of the D10, D50 and D90 values, i.e., the specification of the particle size ranges that comprise 10, 50 or 90% by weight of all powder particles.

The fat absorption capacity (here the absorption capacity of rapeseed oil) and the water absorption capacity relate to the swelling capacity of the powders and are specified in g fat or g water per g powder.

The lightness color value L* and the two color coordinates a* and b* belong to the so-called L*a*b* color system, which is also known as the CIELAB system. It is the most commonly used system for color measurement today and is widely used in almost all areas of application. It was defined by the CIE in 1976 as one of the equidistant color spaces in order to counter the main problem of the original Yxy system: equal geometric difference distances in the x, y color triangle do not lead to equal color differences in terms of sensitivity. The color space of the L*a*b* system is characterized by the brightness L* and the color coordinates a* and b*. The signs indicate the color direction: +a* indicates a red component, while −a* towards green. Accordingly, +b* stands for yellow and −b* for blue. The origin of the coordinates (axis intersection point) is a neutral gray without any chromaticity. With increasing a*b* values, i.e. the further away the color location is from the center, the greater the chromaticity. The CR 400 or 410 device has proven itself for determining the color values in the context of the method according to the invention. Further information can be found in the consumer information “Exact color communication” from Konica Minolta.

Preferably, the set of test parameters should contain at least 5, preferably at least 7 parameters.

### Principal Component Analysis (PCA)

In the next step, the data pool obtained in this way is analyzed using so-called “principal component analysis” (PCA).

This is an established statistical method for analyzing large data sets with a high number of parameters per observation, for improving the interpretability of data while retaining a maximum amount of information and for visualizing multidimensional data. In formal terms, PCA is a statistical technique for reducing the dimensionality of a data set. This is done by a linear transformation of the data into a new coordinate system in which (most of) the variation in the data can be described with fewer dimensions than in the original data. In the method according to the invention, only the first two principal components are used to represent the data in two dimensions and to visually identify clusters of closely related data points.

In PCA data analysis, the first principal component of a set of p variables, which are assumed to be jointly normally distributed, becomes the derived variable, which is formed as a linear combination of the original variables and explains the largest variance. The second principal component explains the largest variance in what remains when the effect of the first component is removed, and we can continue with p iterations until all the variance is explained. PCA is most commonly used when many of the variables are highly correlated with each other and it is desirable to reduce their number to an independent set. Accordingly, the first or second principal component can be defined as a direction that maximizes the variance of the projected data. PCA is the simplest of the true eigenvector-based multivariate analyses and is closely related to factor analysis. A detailed description of the method can be found, for example, in https://en.wikipedia.org/wiki/Principal_component_analysis

Briefly summarized, PCA provides a coordinate system in which the component with the largest variance or deviation (“first component”) is plotted against the component with the second largest variance (“second component”) for each data point. In the final step, each of the data points in the coordinate system is now assigned the respective previously determined product property.

Surprisingly, it was found that this results in a cluster formation that can be clearly correlated with the properties and functionalities to be investigated. The properties and functionalities that can be investigated or predicted in this way include texture, creaminess, synergy effects as well as combinations of two, three or more of these properties.

This means that an unknown raw material sample only needs to be analyzed for the test parameters mentioned at the beginning in order to be able to assign a coordinate to it with the help of PCA. Based on the position of the coordinate, the expected raw material functionality in the final formulation can then be inferred quickly, reliably and, if necessary, automatically. An even faster simulation based on the initial values and future prediction and selection criteria for raw material selection by AI (artificial intelligence) in an even more accelerated approach is therefore easily conceivable and realizable as a second stage. Furthermore, an early and fast selection of suitable alternative raw materials is possible in any industrial implementation.

### EXAMPLES

### Example 1A/B

### Selection of Parameters and Data Generation

In the following, the method according to the invention is explained on the basis of the evaluation of various plant-based powders. For this purpose, the protein fraction was first extracted from the fruits of various plants (pea, field bean, chickpea, oat) and then brought into a powdery state. The following parameters were then selected from those available:
pH value
solubility
DSC data (on set, end set, peak, peak height, reaction enthalpy)
particle size distribution (D10, D50, D90, specific surface area)
fat absorption capacity (rapeseed oil)
water absorption capacity
color coordinates (L*, a*, b*)
   and the corresponding measured values were determined; these are summarized in Table 1.
   The various samples of the plant-based powders were then incorporated into a standard formulation for a vegan plant-based quark and the texture of the resulting products was determined. The results can also be found in Table 1, last column.

TABLE 1A
Data set for quality assessment

| | | | | On set | End set |
|---|---|---|---|---|---|
| Ex. | Origin | pH* | Solubility* | [° C.] | [° C.] |
| 1 | pea | 7.77 | 0.4 | 56.275 | 70.7 |
| 2 | pea | 7.7 | 0.3 | 52.65 | 72.485 |
| 3 | pea | 7.77 | 0.325 | 52.825 | 70.12 |
| 4 | pea | 7.62 | 0.375 | 52.44 | 69.49 |
| 5 | pea | 7.98 | 0.4 | 53.27 | 71.08 |
| 6 | pea | 7.76 | 0.375 | 59.37 | 68.31 |
| 7 | pea | 7.39 | 0.2 | 55.54 | 66.67 |
| 8 | pea | 7.67 | 0.275 | 51.74 | 68.445 |
| 9 | field bean | 7.21 | 0.4 | 55.18 | 66.86 |
| 10 | field bean | 7.08 | 0.25 | 54.27 | 70.105 |
| 11 | chickpea | 7.76 | 0.4 | 56.555 | 73.115 |
| 12 | oat | 7.74 | 0.3 | 53.505 | 69.175 |

| | Peak | Reaction enthalpy | Peak height | Specific surface |
|---|---|---|---|---|
| Ex. | [° C.] | [J/g] | [mW] | [cm²/cm³] |
| 1 | 63.46 | −0.585 | 0.02963 | 2029.9 |
| 2 | 64.965 | −2.265 | 0.103005 | 2359.45 |
| 3 | 62.64 | −1.305 | 0.05987 | 1386.25 |
| 4 | 60.47 | −0.69 | 0.048685 | 1021.1 |
| 5 | 62.725 | −1.095 | 0.069085 | 1316.45 |
| 6 | 63.065 | −1.345 | 0.080835 | 1130.9 |
| 7 | 61.425 | −0.775 | 0.09297 | 545.137705 |
| 8 | 60.94 | −1.71 | 0.11 | 814.445 |
| 9 | 61.375 | −0.305 | 0.02612 | 1756 |
| 10 | 63.055 | −4.64 | 0.185 | 7011.15 |
| 11 | 64.875 | −1.42 | 0.06824 | 1654.15 |
| 12 | 62.065 | −0.97 | 0.04052 | 1371.2 |

*1 wt.-percent solution

TABLE 1B
Data set for quality assessment

| Ex. | Origin | D10 [μm] | D50 [μm] | D90 [μm] |
|---|---|---|---|---|
| 1 | pea | 16.41019 | 36.679675 | 73.50104 |
| 2 | pea | 16.04886 | 28.1959 | 50.241605 |
| 3 | pea | 24.950615 | 49.37172 | 121.441215 |
| 4 | pea | 31.059785 | 72.287075 | 183.973495 |
| 5 | pea | 27.636675 | 51.3814 | 98.7448 |
| 6 | pea | 31.891985 | 59.106525 | 117.480105 |
| 7 | pea | 24.270895 | 98.484615 | 204.588875 |
| 8 | pea | 36.621365 | 117.186405 | 222.080115 |
| 9 | field bean | 19.44992 | 41.04005 | 84.19119 |
| 10 | field bean | 5.51351 | 8.9755 | 15.646215 |
| 11 | chickpea | 20.88119 | 42.092875 | 87.612445 |
| 12 | oat | 24.35504 | 51.666535 | 119.96077 |

| Ex. | FA** | WA*** | L* | a* | b* | Texture |
|---|---|---|---|---|---|---|
| 1 | 0.695 | 3.295 | 82.4 | 2.89 | 19.36 | structured |
| 2 | 0.61 | 1.2 | 85.3 | 0.77 | 25.19 | not structured |
| 3 | 0.66 | 2.775 | 80.68 | 2.8 | 18.17 | structured |
| 4 | 1.005 | 2.49 | 82.32 | 4.24 | 21.51 | gum-like |
| 5 | 0.45 | 2.46 | 79.85 | 3.67 | 19.15 | structured |
| 6 | 0.63 | 2.34 | 81.41 | 3.49 | 18.9 | structured |
| 7 | 0.785 | 1.155 | 77.18 | 6.08 | 19.02 | not structured |
| 8 | 0.86 | 1.155 | 75.76 | 7.32 | 19.94 | not structured |
| 9 | 0.63 | 2.27 | 87.52 | 0.46 | 13.18 | mealy |
| 10 | 0.945 | 1.365 | 89.53 | 0.54 | 13.05 | not structured |
| 11 | 0.68 | 2.91 | 83.79 | 1.42 | 16.25 | structured |
| 12 | 0.79 | 1.505 | 78.96 | 3.01 | 17.21 | dull, mealy |

**Fat absorption [g fat/g powder]
***Water absorption [g water/g powder]

### Example 1C

### Checking the Correlation of the Data Using PCA=Principal Component Analysis

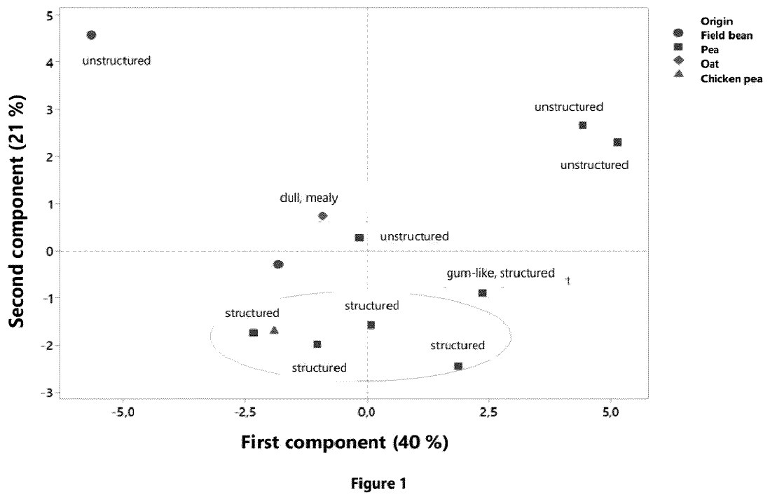

The third step of the method according to the invention is shown in FIG. 1. The previously obtained data set is processed with the aid of principal component analysis. The PCA provides a coordinate system in which the scores for the first two principal components are plotted for each data point. In FIG. 1, the explained variance of principal component 1 is 40.8%. That of the second principal component is 26.1%. Overall, the PCA explains 66.9% of the variance of the original data. Knowing which parameters characterize the two principal components is irrelevant for the procedure, as this step is only concerned with bringing the variance to a scalable size.

As can be seen from the diagram, the data points are distributed more or less evenly across the coordinate system. After assigning the textures actually found to the data points, however, it becomes apparent that the diagram contains clusters of the same or similar structure. Thus, the desired structured textures are found exclusively in the x-coordinate range −2.5 to 2.5 and y-coordinate range +1 to +3. Samples that are not in this cluster were not structured; here the texture ranged from mealy, creamy to rubbery.

FIG. 1 can therefore be understood as a raw material selection diagram. In order to be able to predict the expected functional properties of a raw material, it is only necessary to record the physicochemical data according to the selected test parameters-which are usually either given by the specification anyway or are recorded during the incoming inspection—and to calculate the corresponding data point as described above. The position of the data point in the diagram can then be used to draw conclusions about the raw material functionality to be expected in the end product.

### Example 2

In the following, the method according to the invention is explained on the basis of the evaluation of 17 different milk protein powders;

The following parameters were selected from those available:
pH value
solubility
DSC data (on set, end set, peak, peak height, reaction enthalpy)
particle size distribution (D10, D50, D90, specific surface area)
fat absorption capacity (rapeseed oil)
water absorption capacity
color coordinates (L*, a*, b*)
   and the corresponding measured values were determined; these are summarized in Table 2.

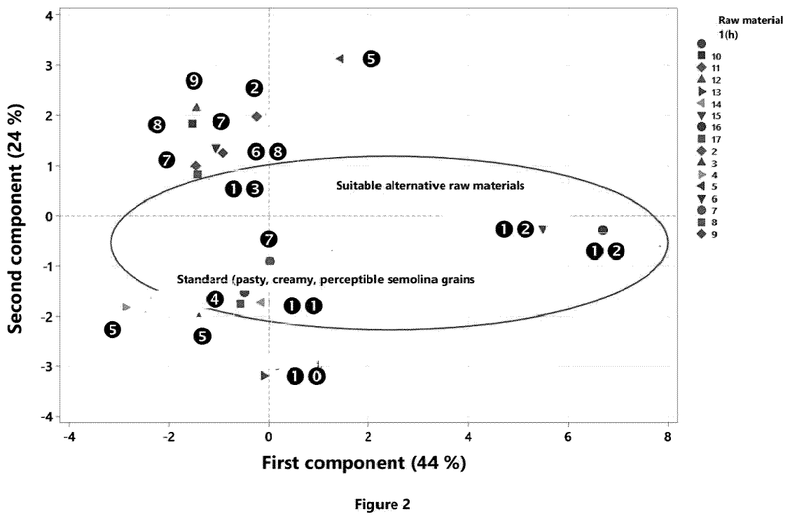

The various samples of milk protein powder were then incorporated into a standard formulation for milk semolina with a high protein content and the texture of the resulting products was determined. The results can also be found in Table 2, last column. PCA was then carried out according to example 1C. The score plot with the cluster of substances with desired properties can be found in FIG. 2.

TABLE 2A
Data set for quality assessment

| Ex. | pH* | Solubility* | On set [° C.] | End set [° C.] |
|---|---|---|---|---|
| 1 | 7.78 | 0.30 | 0.00 | 0.00 |
| 2 | 7.85 | 0.48 | 60.93 | 72.87 |
| 3 | 7.38 | 0.65 | 0.00 | 0.00 |
| 4 | 7.83 | 0.65 | 0.00 | 0.00 |
| 5 | 7.84 | 0.40 | 61.21 | 66.48 |
| 6 | 7.85 | 0.40 | 52.84 | 59.18 |
| 7 | 7.60 | 0.50 | 0.00 | 0.00 |
| 8 | 7.68 | 0.60 | 0.00 | 0.00 |
| 9 | 7.61 | 0.50 | 61.51 | 67.92 |
| 10 | 7.75 | 0.63 | 61.35 | 77.76 |
| 11 | 7.47 | 0.40 | 56.88 | 72.93 |
| 12 | 7.88 | 0.65 | 62.19 | 71.66 |
| 13 | 7.57 | 0.30 | 0.00 | 0.00 |
| 14 | 7.82 | 0.40 | 0.00 | 0.00 |
| 15 | 7.73 | 0.05 | 0.00 | 0.00 |
| 16 | 7.74 | 0.01 | 0.00 | 0.00 |
| 17 | 7.91 | 0.15 | 58.15 | 61.80 |

TABLE 2B
Data set for quality assessment

| Ex. | Peak [° C.] | Specific surface [cm²/cm³] |
|---|---|---|
| 1 | 0.00 | 7.78 |
| 2 | 64.35 | 7.85 |
| 3 | 0.00 | 7.38 |
| 4 | 0.00 | 7.83 |
| 5 | 64.03 | 7.84 |
| 6 | 55.63 | 7.85 |
| 7 | 0.00 | 7.60 |
| 8 | 0.00 | 7.68 |
| 9 | 67.92 | 7.61 |
| 10 | 66.28 | 7.75 |
| 11 | 66.29 | 7.47 |
| 12 | 66.83 | 7.88 |
| 13 | 0.00 | 7.57 |
| 14 | 0.00 | 7.82 |
| 15 | 0.00 | 7.73 |
| 16 | 0.00 | 7.74 |
| 17 | 60.36 | 7.91 |

TABLE 2C
Data set for quality assessment

| Ex. | D10 [μm] | D50 [μm] | D90 [μm] | FA** | WA*** |
|---|---|---|---|---|---|
| 1 | 23.98 | 55.87 | 167.72 | 2.23 | 1.37 |
| 2 | 35.52 | 86.49 | 222.32 | 2.36 | 3.64 |
| 3 | 23.04 | 61.05 | 168.18 | 2.23 | 4.42 |
| 4 | 17.70 | 41.80 | 88.29 | 2.30 | 4.65 |
| 5 | 31.96 | 171.26 | 322.69 | 1.31 | 3.62 |
| 6 | 27.75 | 69.27 | 186.03 | 2.08 | 3.22 |
| 7 | 26.17 | 93.50 | 250.93 | 1.73 | 3.73 |
| 8 | 25.48 | 62.62 | 193.51 | 2.42 | 4.53 |
| 9 | 33.96 | 74.92 | 225.91 | 2.60 | 3.55 |
| 10 | 27.10 | 69.60 | 197.09 | 2.36 | 3.89 |
| 11 | 21.24 | 64.05 | 190.68 | 2.39 | 3.98 |
| 12 | 28.66 | 71.08 | 207.07 | 1.58 | 3.19 |
| 131 | 25.64 | 53.25 | 102.00 | 3.59 | 2.25 |
| 14 | 28.19 | 65.22 | 185.75 | 2.62 | 3.19 |
| 15 | 42.96 | 175.53 | 368.09 | 2.09 | 0.30 |
| 16 | 58.08 | 187.30 | 392.03 | 2.64 | 0.14 |
| 17 | 18.25 | 50.40 | 111.51 | 2.14 | 1.15 |

* 1% solution
**Fat absorption [g fat/g powder]
***Water absorption [g water/g powder]

TABLE 2D
Data set for quality assessment

| Ex. | L* | a* | b* | Texture |
|---|---|---|---|---|
| 1 | 93.34 | −1.21 | 9.14 | (1) paste-like, creamy, perceptible |
| | | | | semolina grains |
| 2 | 91.91 | −0.74 | 9.92 | (2) less firm than standard, creamy |
| 3 | 92.75 | −1.14 | 9.88 | (3) too solid/compact |
| 4 | 94.18 | −1.71 | 9.42 | (4) comparable to standard |
| 5 | 92.13 | −1.34 | 12.29 | (5) too solid/compact |
| 6 | 92.54 | −1.26 | 9.35 | (6) firmer and stickier than standard |
| 7 | 92.75 | −1.14 | 9.88 | (7) minimally firmer than standard; ok |
| 8 | 92.12 | −1.36 | 12.65 | (8) minimally too thin/watery |
| 9 | 92.98 | −1.80 | 10.75 | (8) minimally too thin/watery |
| 10 | 92.47 | −0.28 | 7.77 | (8) minimally too thin/watery |
| 11 | 92.50 | −0.69 | 9.35 | (7) minimum firmer than standard |
| 12 | 93.18 | −2.12 | 11.92 | (9) firmer than standard |
| 131 | 90.93 | −1.65 | 12.86 | (10) firmer and stickier than standard |
| 14 | 92.29 | −1.84 | 12.10 | (11) comparable to standard, minimally |
| | | | | creamier |
| 15 | 88.26 | −0.57 | 13.74 | (12) slightly softer than standard.; ok |
| 16 | 87.71 | −0.37 | 13.87 | (12) slightly softer than standard.; ok |
| 17 | 93.39 | −2.34 | 12.61 | (13) ok |

*1% solution
** Fat absorption [g fat/g powder]
*** Water absorption [g water/g powder]

1. A method for predicting raw material functionality in end products such as dairy products, primary and main milk-based products including cheese, their plant-based milk alternatives and corresponding hybrid products, comprising the following steps
(a) providing a set of raw materials of different qualities;
(b) establishing a set of test parameters;
(c) applying the set of test parameters to each of the individual samples from step (a) to generate a data set;
(d) incorporating the raw materials of different qualities from step (a) into final products, such as dairy products, products based primarily and principally on milk, including cheeses whose plant-based milk alternatives and corresponding hybrid products;
(e) recording and evaluation of product characteristics;
(f) checking the correlation of the data in the data set using Principal Component Analysis (PCA) to obtain a coordinate system (score plot) and determining the largest variance of the data as the first component and the second largest variance of the data as the second component;
(g) assigning the product properties found from step (e) to the data points in the coordinate system from step (f); and
(h) determining clusters in the score plot to identify raw materials which, when used in the end product, lead to the desired similar or identical product properties.

2. The method of claim 1, wherein the end products used are milk products, products based primarily and mainly on milk, including cheeses, their plant-based milk alternatives and corresponding hybrid products, which are selected from the group formed by whole milk, skimmed milk, UHT milk, milk powders, cream, curd, cheese and yoghurt, and cheese and their plant-based substitute products and applications such as, among others including plant-based drinks, spreads, cream alternatives, yoghurt alternatives and cheese alternatives.

3. The method of claim 2, wherein milk- and/or plant-based raw materials/ingredients/ingredients are used which are selected from the group formed by proteins, fats, carbohydrates, acid whey, milk permeates and milk retentates as well as mixtures and concentrates thereof.

4. The method of claim 1, wherein raw material sample set comprising 5 to 50 samples is used.

5. The method of claim 1, wherein sample set is used which comprises raw materials of at least three different qualities.

6. The method of claim 1, wherein a set of test parameters is used which comprises at least two of the following parameters:
pH value;
solubility;
thermal behavior (DSC) of the powder;
particle size distribution (D10, D50, D90, specific surface area);
fat absorption capacity (rapeseed oil);
water absorption capacity;
dissolved oxygen quantity;
instability index (Lumisizer);
lightness color value white/black (L*);
color coordinate red/green (a*);
color coordinate yellow/blue (b*);
free amino nitrogen (PAN);
ammonia content;
urea content;
calcium content;
acidity;
rheological behavior;
firmness; and
degree of syneresis.

7. The method of claim 6, wherein a set of test parameters is used which comprises at least 5.

8. The method of claim 1, wherein properties are predicted which are selected from the group formed by texture, creaminess, syneresis, mouthfeel, firmness, emulsion stability, synergy effects as well as combinations of two, three or more of these properties and prediction of their application properties.

9. The method of claim 7, wherein the set of test parameters comprises at least 7 parameters.
